(19) United States
(12) Patent Application Publication
(10) Pub. No.: US 2018/0193605 A1
(43) Pub. Date: Jul. 12, 2018

(54) CATHETER ASSEMBLY FOR DELIVERING A MEDICAL DEVICE

(71) Applicant: ABBOTT CARDIOVASCULAR SYSTEMS INC., Santa Clara, CA (US)

(72) Inventors: Daniel H. Shumer, San Jose, CA (US); Ronald G. Earles, Houston, TX (US); Nianjiong J. Bei, Palo Alto, CA (US); Barbara Stamberg, San Jose, CA (US); Daniel C. Simon, Murrieta, CA (US); Maria Del Rosario Nava, Baldwin Park, CA (US)

(73) Assignee: ABBOTT CARDIOVASCULAR SYSTEMS INC., Santa Clara, CA (US)

(21) Appl. No.: 15/836,649

(22) Filed: Dec. 8, 2017

Related U.S. Application Data
(60) Provisional application No. 62/497,929, filed on Dec. 8, 2016.

(51) Int. Cl. A61M 25/06; A61M 25/00; A61M 25/01

(52) U.S. Cl. CPC A61M 25/0662; A61M 25/0068; A61M 25/0136; A61F 2/966; A61M 2025/015; A61M 2025/0681; A61M 25/005

(57) ABSTRACT

Catheter assembly for delivering an implant including an outer tubular member, an inner shaft member, and a pusher assembly. The outer tubular member defines an outer tubular member lumen and includes an inner layer, a reinforcement layer, a middle layer, and an outer layer. The inner shaft member is disposed at least partially within the outer tubular member lumen. The inner shaft member includes a proximal inner shaft portion and a distal inner shaft portion, the distal inner shaft portion having a distal end portion. The pusher assembly is coupled to the distal end portion of the distal inner shaft portion. The inner shaft member is configured to move distally and proximally relative the outer tubular member between an initial position and a deployed position.

### CROSS-REFERENCE TO RELATED APPLICATIONS

This application claims the benefit of U.S. Provisional Patent Application No. 62/497,929, filed Dec. 8, 2016, which is incorporated herein by reference in its entirety.

### BACKGROUND

### Field of Disclosed Subject Matter

The disclosed subject matter is directed to a catheter assembly, and related methods and systems, for delivering one or more medical devices, such as a braided implant. The braided implant, for example a stent or scaffold, can be disposed within a catheter assembly, which is configured to deliver the braided implant by reciprocally moving an inner shaft member distally and proximally relative an outer tubular member.

### Description of Related Art

Conventional self-expanding stent delivery systems can include a handle housing portion and an elongated shaft (e.g., a catheter assembly), wherein the stent is disposed within a delivery portion at the distal end of the shaft. To deploy the stent, an outer sheath typically is retracted relative to the stent, whereby the stent is exposed and released from its delivery configuration. In certain systems, an inner member having a pushing mechanism can be used push the stent from the outer sheath, while the outer sheath is retracted.

However, certain self-expanding implants, such as braided stents, experience excessive elongations when compressed to a delivery condition. Such configurations introduce unique challenges for delivery and deployment. As such, there remains a need for a catheter assembly, and related system and method, for delivering an implant, such as a braided stent, using a relatively simple motion and ease of use. Furthermore, there is a need for such a delivery system capable of being secured in a fixed position during activation, and having an outer profile less than or equal to 6 French.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to a catheter assembly, and related systems and methods, for delivering a medical device. For example, the system for delivering a medical device can include a handle, a trigger operatively coupled to the handle, an actuation assembly operatively coupled to the trigger, and a catheter assembly.

In accordance with the disclosed subject matter, the catheter assembly includes an outer tubular member, an inner shaft member, and a pusher assembly. The outer tubular member defines an outer tubular member lumen and includes an inner layer, a reinforcement layer, a middle layer, and an outer layer. The inner shaft member is disposed at least partially within the outer tubular member lumen and includes a proximal inner shaft portion and a distal inner shaft portion. The distal inner shaft portion includes a distal end portion. The pusher assembly is coupled to the distal end portion of the distal inner shaft portion. The inner shaft member is configured to move distally and proximally relative the outer tubular member between an initial position and a deployed position.

The inner layer of the outer tubular member can be fluorinated ethylene propylene. The reinforcement layer of the outer tubular member can be a stainless steel braid. The reinforcement layer of the outer tubular member can be Teflon fibers. The middle layer of the outer tubular member can be polyimide. The outer layer of the outer tubular member can be Grilamid.

The outer tubular member further can include an atraumatic distal tip having a distally tapered end. The atraumatic distal tip can be heat bonded to the outer tubular member. The atraumatic distal tip can be mounted to an outer diameter of the outer tubular member. The atraumatic distal tip can be polyether block amide. The implant can be disposed within the outer tubular member lumen proximate the pusher assembly.

The catheter assembly can include a stabilizer member having a stabilizer lumen defined therethrough. The stabilizer lumen can have an inner diameter sized to receive the outer tubular member therein. The outer tubular member can be configured to rotate about a central longitudinal axis relative to the stabilizer member. The stabilizer member can include an inner layer, a reinforcement layer, a middle layer, and an outer layer. The stabilizer member can have a distal end portion having an atraumatic tip.

The catheter assembly can include a strain relief coupled to a proximal end portion of the stabilizer member. The stabilizer member can be configured to rotate about a central longitudinal axis relative the strain relief. The catheter assembly can have an outer profile less than or equal to 6 French. For example, the outer profile can be 5 French, 4 French, 3 French, or any suitable increment therebetween. Alternatively, the outer profile can be greater than 6 French. For example, the outer tubular member can be 7 French, 10 French, 15 French, or any suitable increment therebetween.

At least one of the proximal inner shaft portion and the distal inner shaft portion can include an inner layer, a reinforcement layer, and an outer layer. The distal inner shaft portion can be a distal inner shaft member and the proximal inner shaft portion can be a proximal inner shaft member coupled to the distal inner shaft member. A proximal end portion of the distal inner shaft member can be heat bonded to a distal end portion of the proximal inner shaft member. A proximal end portion of the distal inner shaft portion can be an inner taper. An outer diameter at a proximal end portion of the distal inner shaft member can be sized to be received within an inner diameter at a distal end portion of the proximal inner shaft member.

A support tube can be disposed within the outer tubular member lumen. A support coil can be disposed within the outer tubular member lumen distal of the support tube. The pusher assembly can include a stem coupled to the distal end portion of the distal inner shaft portion and an implant-engaging member extending from the stem. A guide wire lumen can be coupled to a distal end portion of the stem and can extend distally of the outer tubular member. The guidewire lumen can include at least one radiopaque marker. A ratchet rack can be coupled to a proximal end portion of the proximal inner shaft portion.

A hypotube can be disposed at least partially within an inner shaft member lumen defined by the inner shaft member. A polymer sleeve can be secured to a distal end portion of the hypotube. A luer can be coupled to a proximal end portion of the hypotube. The inner shaft member can be configured to rotate about a central axis relative to the actuation assembly.

In accordance with the disclosed subject matter a deliver system for delivering an implant can include a handle, a trigger operatively coupled to the handle, an actuation assembly operatively coupled to the trigger, and a catheter assembly operatively coupled to the actuation assembly. The catheter assembly can include an outer tubular member, an inner shaft member, and a pusher assembly. The outer tubular member defines an outer tubular member lumen and includes an inner layer, a reinforcement layer, a middle layer, and an outer layer. The inner shaft member is disposed at least partially within the outer tubular member lumen and includes a proximal inner shaft portion and a distal inner shaft portion. The distal inner shaft portion includes a distal end portion. The pusher assembly is coupled to the distal end portion of the distal inner shaft portion. The inner shaft member is configured to move distally and proximally relative the outer tubular member between an initial position and a deployed position. The actuation assembly can be configured to displace the outer tubular member in the proximal direction a first distance (d) relative to the handle and to separately move the inner shaft member distally a second distance (x) relative to the handle upon deployment of the trigger from a first position to a second position, and further wherein the actuation assembly can be configured to move the inner shaft member proximally a third distance (y) relative to the handle with no displacement of the outer tubular member relative to the handle upon return of the trigger from the second position to the first position. The delivery system can include a stabilizer member and the catheter assembly can have an outer profile less than or equal to 6 French.

In accordance with the disclosed subject matter, a method of forming a catheter assembly for delivering a medical device can include forming an outer tubular member having an inner layer, a reinforcement layer, a middle layer, and an outer layer, providing, and inserting into the outer tubular member, an inner shaft member comprising a proximal inner shaft portion and a distal inner shaft portion, each of the proximal inner shaft portion and distal inner shaft portion comprising an inner layer, a reinforcement layer, and an outer layer, and providing, and positioning about the outer tubular member, a stabilizer member having an inner layer, a reinforcement layer, a middle layer, and an outer layer.

Forming the outer tubular member can include forming the outer tubular member by a coating process. Providing the stabilizer member can include forming the stabilizer member by a coating process. Providing the inner shaft member can include forming the stabilizer member by a coating process.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the disclosed subject matter. Together with the description, the drawings serve to explain the principles of the disclosed subject matter.

### BRIEF DESCRIPTION OF THE FIGURES

FIG. 1 is a right perspective view as viewed from a front of an exemplary embodiment of a delivery system in accordance with the disclosed subject matter.

FIG. 2 is a left side view, with a portion of the handle housing removed, of the delivery system of FIG. 1.

FIG. 3 is a perspective view of an exemplary embodiment of a catheter assembly in accordance with the disclosed subject matter.

FIG. 4 is a partial cross-sectional view of the catheter assembly of FIG. 3.

FIG. 5 is an enlarged detail view of section 5.

FIG. 6 is an enlarged detail view of section 6.

FIG. 7 is an enlarged detail view of section 7.

FIG. 8 is an enlarged detail view of section 8.

FIG. 9A is an enlarged detail view of section 9 and FIG. 9B is an enlarged perspective detail view of an alternative embodiment of section 9.

FIG. 10 is an enlarged detail view of section 10.

FIGS. 11A-D (collectively FIG. 11) provide cross-sectional view of the outer tubular member (FIG. 11A), proximal inner shaft member (FIG. 11B), distal inner shaft member (FIG. 11C), and stabilizer member (FIG. 11D).

FIG. 12 illustrates the connection between the proximal inner shaft member and the distal inner shaft member prior to bonding.

FIG. 13 illustrates an enlarged side view of a distal end of a hypotube of the delivery system of FIG. 1.

FIG. 14 illustrates a cross-sectional side view of the distal end of the hypotube of FIG. 13.

### DETAILED DESCRIPTION

Reference will now be made in detail to the various exemplary embodiments of the disclosed subject matter, exemplary embodiments of which are illustrated in the accompanying drawings. The structure and corresponding method of making and using the disclosed subject matter will be described in conjunction with the detailed description of the catheter assembly and related delivery system. The catheter assembly, and related methods and systems, described herein can be used for delivering a medical device, such as a stent, scaffold, stent graft, valve, filter, or other suitable implant to a desired location in a patient.

A variety of types of medical devices are suitable for delivery by the catheter assembly and related delivery system of the disclosed subject matter. For purpose of illustration and not limitation, the catheter assembly and related delivery system is described herein with a medical device depicted as a self-expanding stent. Particularly, although not by limitation, reference is made herein to the implant being a braided stent or scaffold for purpose of illustration only. However, the catheter assembly and related delivery system presently disclosed is not limited to the delivery of self-expanding stents. Other devices can also be delivered by the system herein. For example, scaffolds, coils, filters, stent grafts, embolic protection devices, and artificial valves can be delivered within a patient's vasculature, heart, or other organs and body lumens using the disclosed catheter assembly and related delivery system. Other devices such as a prosthesis retrieval mechanism can also be delivered with the catheter assembly and related delivery system to a predetermined location in a patient's luminal system. Moreover, a combination of medical devices and/or beneficial agents can also be delivered using the disclosed subject matter. For example, multiple stents and/or a combination of stents and embolic protection devices and/or beneficial agents can be delivered by the disclosed subject matter, as described below. Additional information related to delivery of implants can be found in U.S. application Ser. No. 11/876,764, filed on Oct. 22, 2007, and U.S. application Ser. No. 13/118,325, filed on May 27, 2011, U.S. application Ser. No. 14/932,848, filed Nov. 4, 2015, U.S. application Ser. No. 14/932,795, filed Nov. 4, 2015, U.S. application Ser. No. 14/932,875, filed Nov. 4, 2015, U.S. application Ser. No. 14/932,862, filed Nov. 4, 2015, U.S. application Ser. No. 14/932,884, filed Nov. 4, 2015, U.S. application Ser. No. 14/932,805, filed Nov. 4, 2015, U.S. application Ser. No. 14/932,830, filed Nov. 4, 2015, and U.S. application Ser. No. 14/932,900, filed Nov. 4, 2015, each of which is incorporated by reference in its entirety herein.

Generally, and as set forth in greater detail below, the disclosed subject matter provided herein includes a delivery system having a handle, a trigger, an actuation assembly, and a catheter assembly. A catheter assembly as disclosed herein includes an outer tubular member, an inner shaft member, and a pusher assembly. The outer tubular member defines an outer tubular member lumen and includes an inner layer, a reinforcement layer, a middle layer, and an outer layer. The inner shaft member is disposed at least partially within the outer tubular member lumen and includes a proximal inner shaft portion and a distal inner shaft portion. The distal inner shaft portion includes a distal end portion. The pusher assembly is coupled to the distal end portion of the distal inner shaft portion. The inner shaft member is configured to move distally and proximally relative the outer tubular member between an initial position and a deployed position.

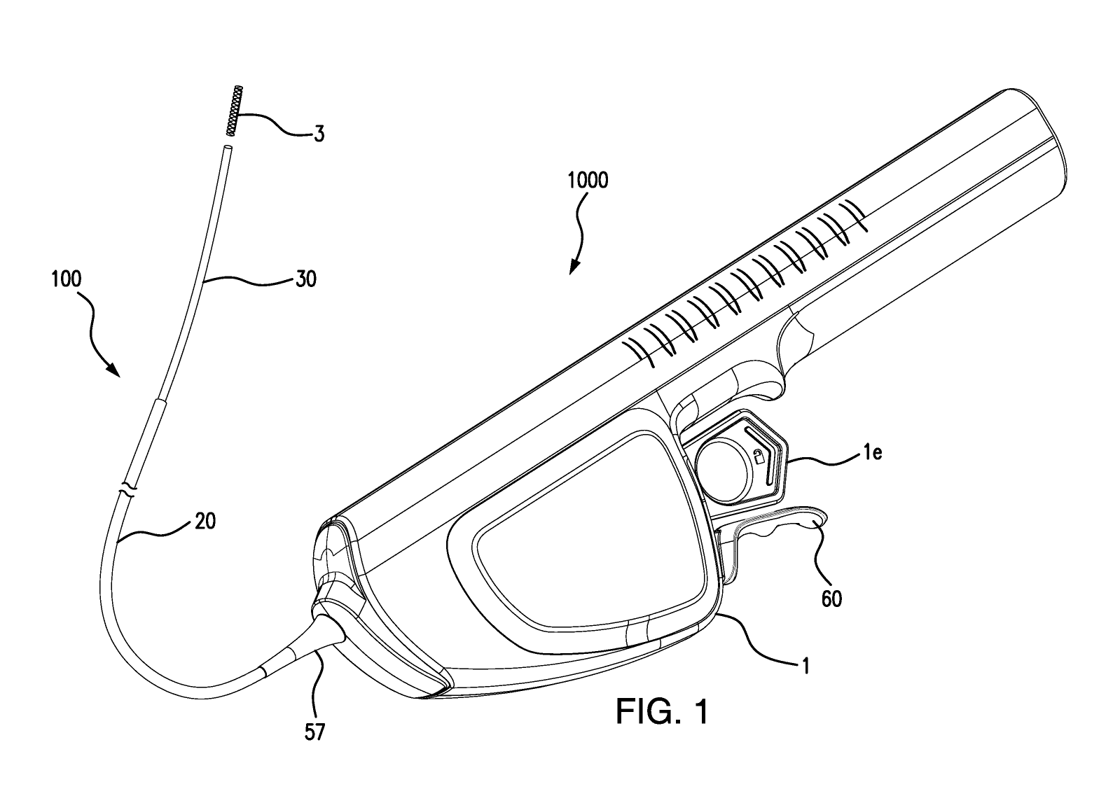

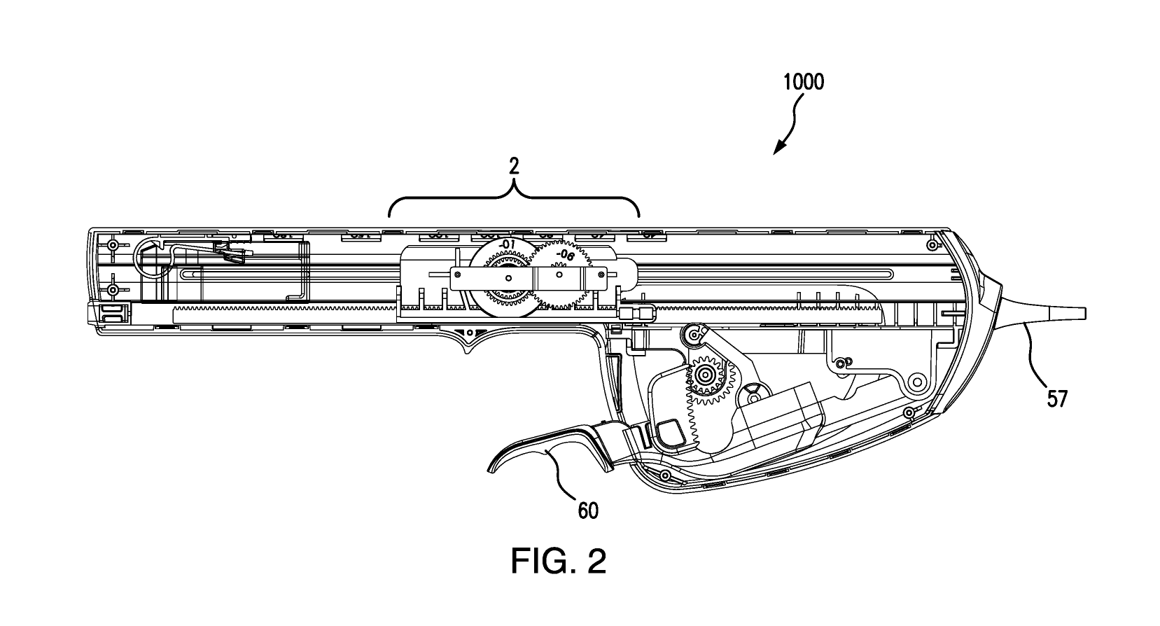

Referring to FIG. 1 for the purpose of illustration and not limitation, various embodiments of the delivery system 1000 disclosed herein generally can include a handle 1, and a catheter assembly 100 (for purpose of clarity all features of catheter assembly 100 as embodied herein are not shown in FIG. 1). For example, the handle 1 as embodied herein can include a trigger assembly including a trigger 60 movable between a first position and a second position, and an actuation assembly 2 (see e.g., FIG. 2) operatively coupled to the trigger 60. A trigger lock le can prevent any motion of the trigger. Additional details of the delivery system, including the actuation assembly, are set forth below.

The catheter assembly of the disclosed subject matter and as embodied herein includes an outer tubular member 20 and an inner shaft member 30 disposed at least partially within the outer tubular member 20. The outer tubular member 20 and the inner shaft member 30 each can be operatively coupled to the actuation assembly 2. The inner shaft member 30 of the disclosed delivery system is movable distally and proximally relative to the outer tubular member 20. The implant 3 can be disposed within the distal end portion of the outer tubular member 20 and positioned to be engaged by the distal end portion of the inner shaft member 30 when the inner shaft member is moved distally relative to the outer tubular member 20. To engage the implant, the distal end portion of the inner shaft member 30 can have a pusher assembly 60 disposed thereon. For example, U.S. application Ser. No. 13/118,325, filed on May 27, 2011, which is incorporated by reference in its entirety herein, discloses suitable pusher elements for the delivery system. The outer tubular member 20 is depicted with a break in FIG. 1 to indicate that the length shown is only exemplary and the catheter assembly 100, including the outer tubular member 20 and inner shaft member 30, can be longer than shown. For example, and not by way of limitation, the outer tubular member 20 can be between 20 and 70 inches long, for example, the outer tubular member 20 can be about 33 or 55 inches long. For example, and not by way of limitation, the inner shaft member 30 can be between 25 and 65 inches long, for example, the inner shaft member 30 can be about 34 or 56 inches long. Indeed, any suitable length can be used. As an example and not by way of limitation, the catheter assembly 100 can be long enough to extend from outside the body of a patient through a tortuous path to a treatment location within the body of a patient.

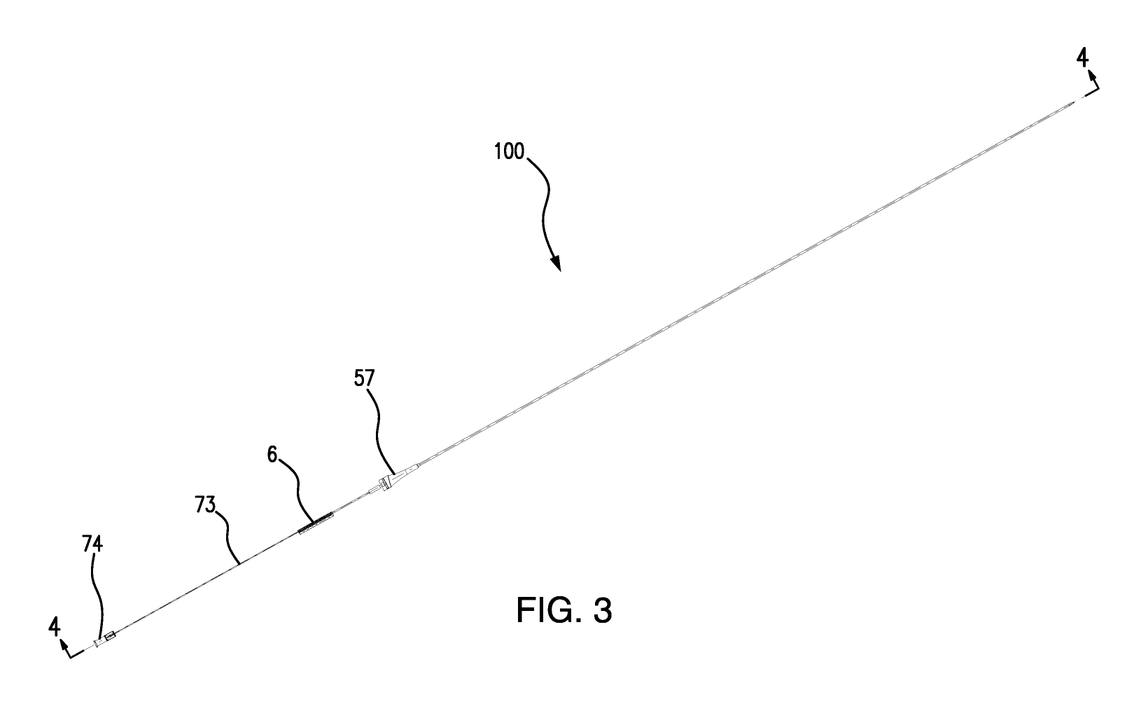

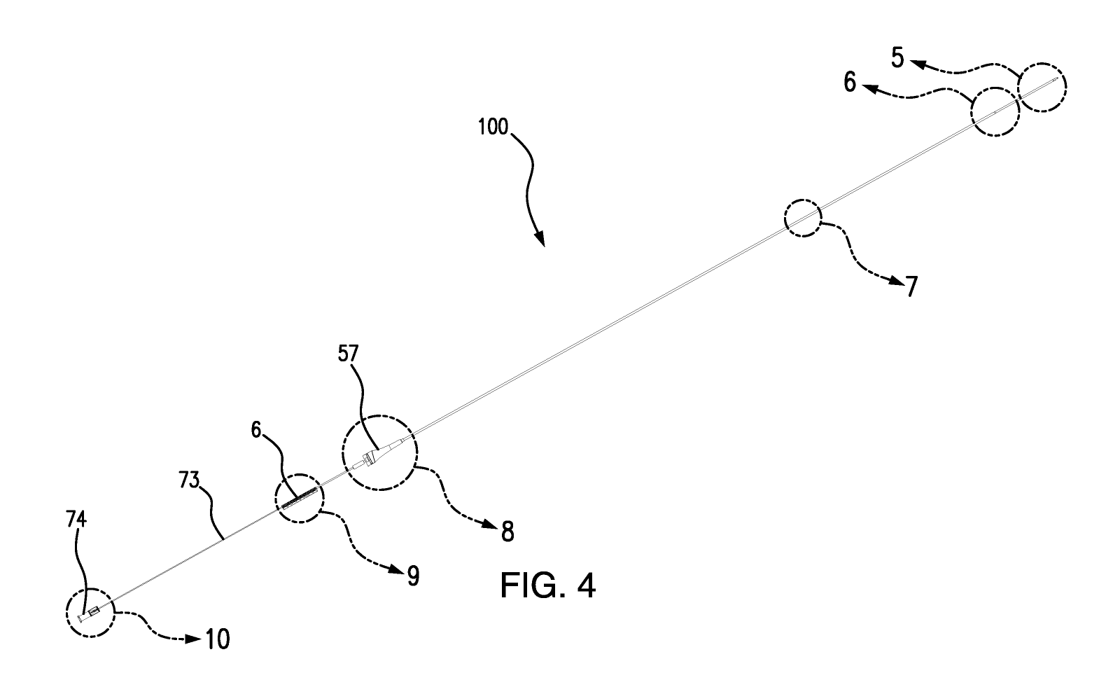

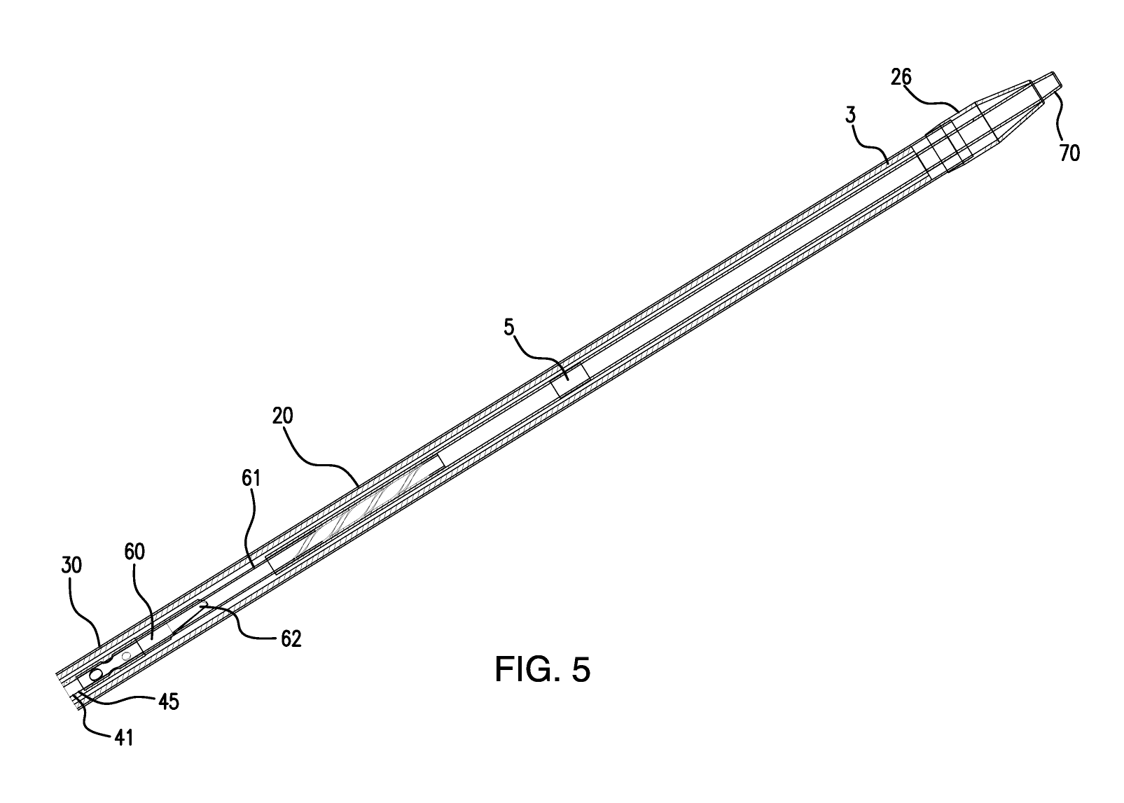

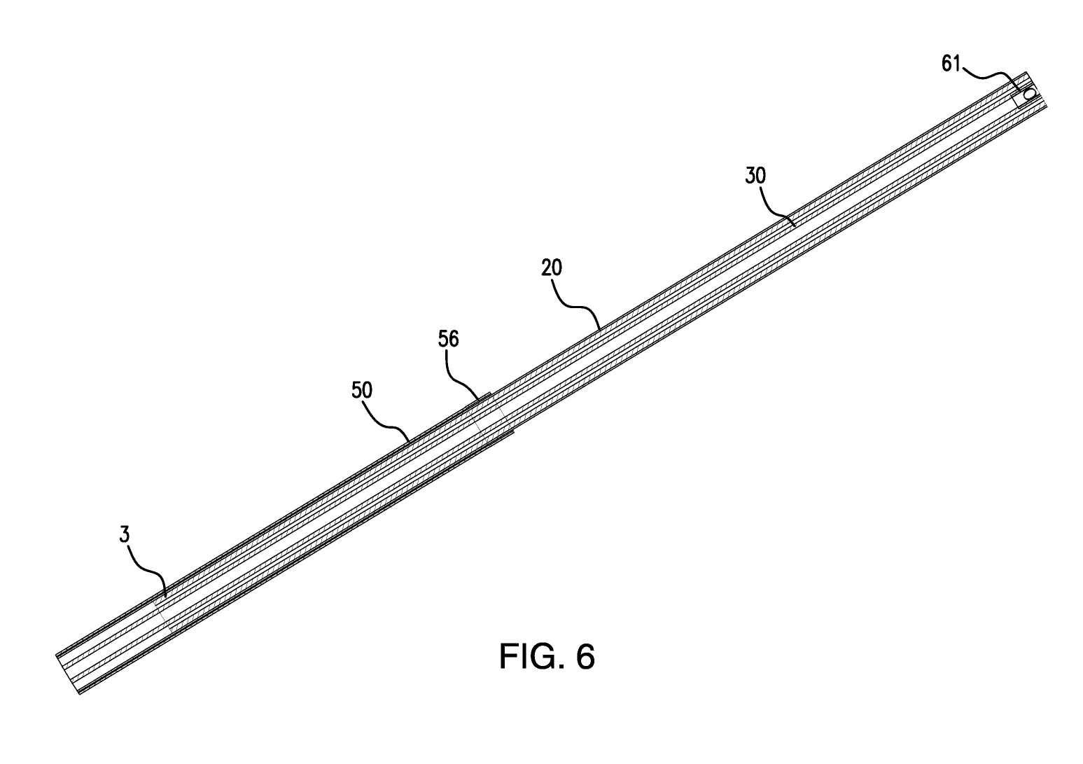

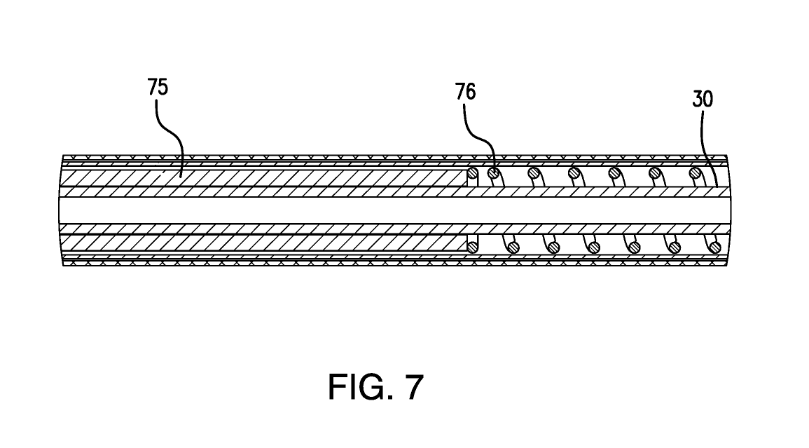

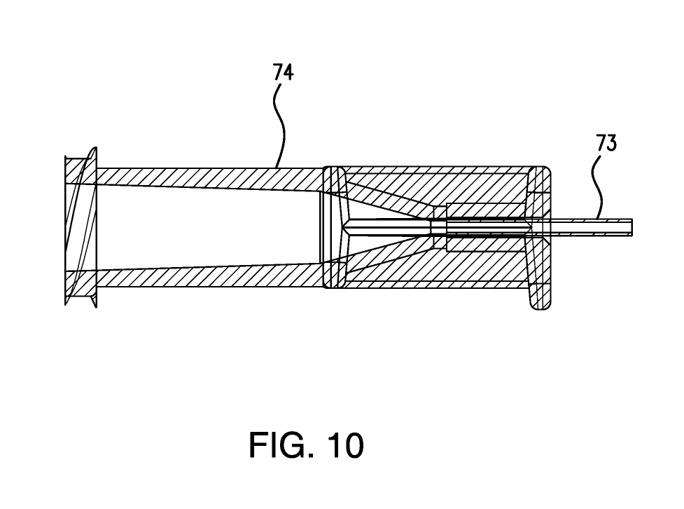

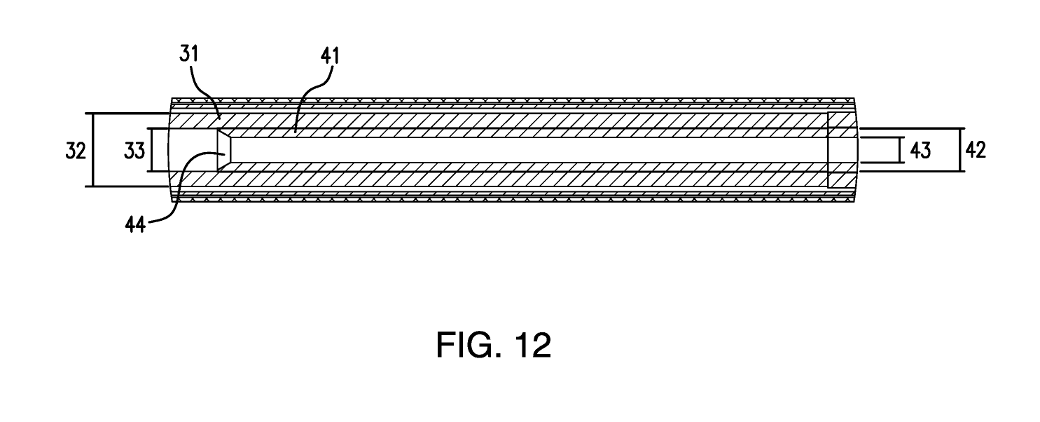

For the purpose of illustration, and not limitation, an exemplary embodiment of a catheter assembly for delivering a medical device is shown in FIG. 3 and is designated generally by reference character 100. Enlarged details of this exemplary embodiment are depicted in FIGS. 4-12. As embodied herein, the catheter assembly includes an outer tubular member 20, an inner shaft member 30, and a pusher assembly 60. Additionally, as described further below, the catheter assembly can further include a stabilizer member 50, defining a stabilizer member lumen 51.

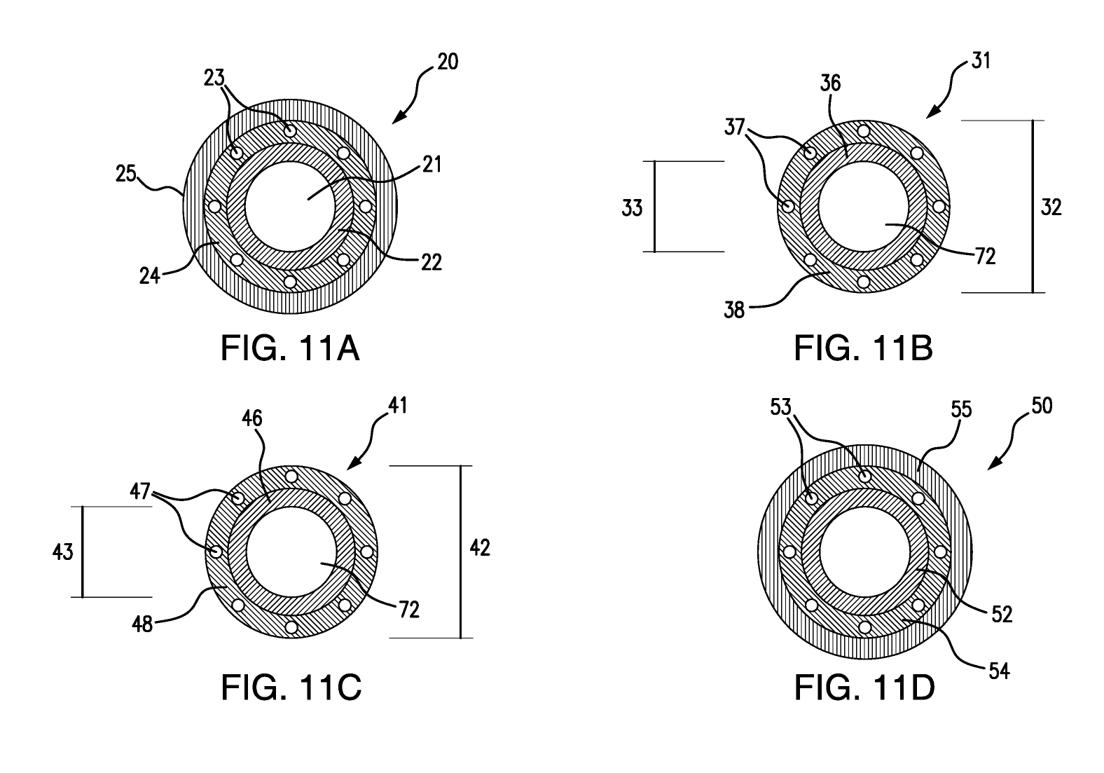

For purpose of illustration and not limitation, the outer tubular member 20 of the disclosed subject matter is a multi-layer tubular member defining an outer tubular member lumen 21. The outer tubular member 20 embodied herein has an inner layer 22, a reinforcement layer 23, a middle layer 24, and an outer layer 25. The inner layer 22 can be fluorinated ethylene propylene. The middle layer 24 can be a polymer, for example, polyimide. The outer layer 25 can be a plastic, for example Nylon 12, such as Grilamid. The reinforcement layer 23 is configured to provide additional strength by providing fibers or wires, such as in the form of a braid around the inner layer 22 or as longitudinally extending fibers. For example, the reinforcement layer 23 can be a braid of stainless steel (SST), for example, 0.001×0.005 inch, 304V SST. The braid can include 16 wires having high tensile strength. Although only eight wires are shown in FIG. 11A, any number of wires can be provided as needed or desired. As another example, the reinforcement layer 23 can include Kevlar fibers extending longitudinally along the length of the outer tubular member.

Such a four-layer configuration thus provides improved hoop strength and flexibility for the outer tubular member while allowing a thinner wall than traditional catheter configurations. For example, with an inner diameter of about 0.0660 inches and a wall thickness of about 0.0078 inches, an outer diameter of about 0.0738 inches can be achieved, such as by a coating process as noted below. By providing a thinner wall than traditional catheters, an additional tubular member (e.g., the stabilizer member 50, as described further below) can be provided with the catheter assembly 100 without increasing the overall outer diameter of the catheter assembly 100 or reducing the hoop strength and flexibility of the catheter assembly 100. For example, a 6 Fr. or less catheter can be achieved, either with or without an additional tubular member (e.g., stabilizer).

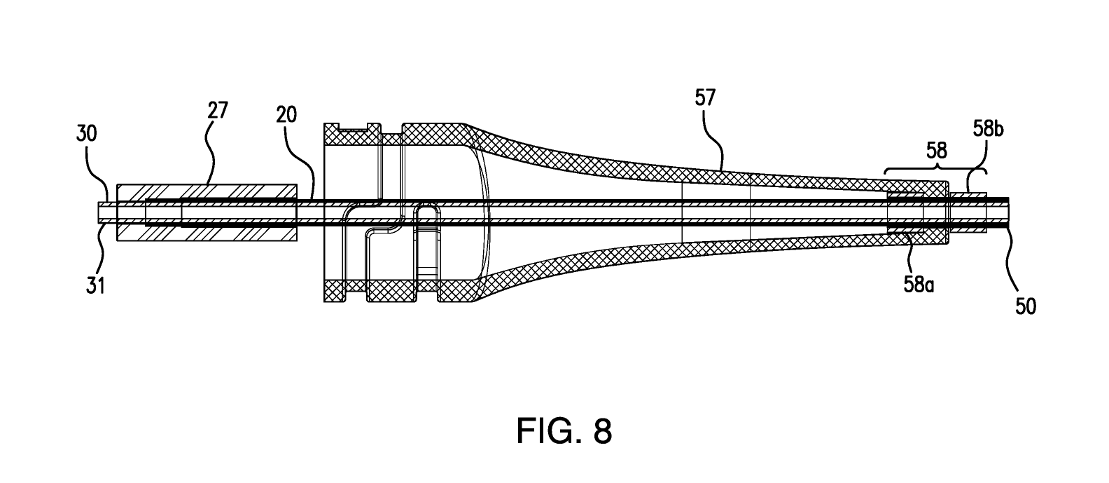

A proximal end portion of the outer tubular member 20 can be coupled directly to the handle in a fixed relation, if desired, or to the actuation assembly if relative movement of the outer tubular member 20 is desired, such as previously described. As embodied herein, the proximal end portion of the outer tubular member 20 can be coupled to the actuation assembly 2 by a cap seal 27 (see FIG. 8). For example, the cap seal 27 can be cylindrical and received within a cavity of the actuation assembly to allow the outer tubular member 20 to rotate about a central longitudinal axis relative the actuation assembly 2. Such a coupling thus can allow the outer tubular member 20 to rotate about a central longitudinal axis, for example, during insertion of the catheter assembly 100 and during delivery of the implant. Alternatively, the cap seal 27 can be shaped to mate or engage the actuation assembly to prevent the outer tubular member 20 from rotating relative the actuation assembly 2.

The outer tubular member 20 can have an atraumatic distal tip 26. The atraumatic distal tip 26 can reduce or prevent damage to vessel walls during delivery of the catheter 100. Additionally, the atraumatic distal tip 26 can be configured to be flexible to allow deployment of the implant 3 through the atraumatic distal tip 26 without splitting thereof. For example, the atraumatic distal tip 26 can be made of Pebax and can be configured with a tapered distal end. The atraumatic distal tip 26 can be a separate member heat bonded to a distal end portion of the outer tubular member 20, for example, the atraumatic distal tip 26 can be heat bonded to an outer diameter of the distal end portion of the outer tubular member 20. Additionally, the atraumatic distal tip 26 can be configured to receive a guidewire therethrough. For example, a guidewire lumen 70 can be disposed at least partially within the outer tubular member lumen 21. The guidewire lumen 70 can be coupled to a distal end of the pusher assembly, as described below, and extend distally of the atraumatic distal tip 26 and out of the outer tubular member 20. The atraumatic distal tip 26 can be flexible enough to collapse onto the guidewire lumen 20. The guidewire lumen 70 can have at least one radiopaque marker 5, such as a band or printed indicia, disposed thereon.

As previously noted, it may be desirable for the catheter assembly of the disclosed subject matter to include a stabilizer 50. For example, and as embodied herein, the delivery system and methods embodied herein can be configured for movement of the outer tubular member 20 relative the handle. The stabilizer provides a location in which to secure position of the delivery system, such as by a hemostatic valve and/or guide catheter, while allowing relative movement of the outer tubular member. The stabilizer member 50 can be a multi-layer tubular member. For example, the stabilizer can have an inner layer 52, a reinforcement layer 53, a middle layer 54, and an outer layer 55. The inner layer 52 can be a synthetic fluoropolymer, for example, polytetrafluoroethylene (PTFE). The middle layer 54 can be a strike layer over the reinforcement layer 53 and can include a polymer, for example, polyimide. The outer layer 55 can be a plastic, for example, Grilimid. The reinforcement layer 53, like the reinforcement layers of the outer tubular member 20 and the inner shaft member 30 can provide additional strength by providing fibers or wires that can be formed as a braid around the inner layer 52 or longitudinally extending fibers. For example, the reinforcement layer 53 can be a braid of stainless steel (SST), for example, 0.0007×0.003 inch, 304V SST. The braid can include 16 wires having high tensile strength. As another example, the reinforcement layer 53 can include Teflon fibers extending longitudinally along the length of the stabilizer member. As with the outer tubular member, such a four-layer configuration can allow for have improved hoop strength and flexibility while having a thinner wall than traditional catheters. Furthermore, and as noted above, by providing tubular members with a thinner wall than traditional catheters, the catheter assembly 100 can include a combination of an outer tubular member and a stabilizer member without increasing the outer diameter of the catheter assembly 100 or reducing the hoop strength and flexibility of the catheter assembly 100. The stabilizer member 50 can be, for example, between 10 and 60 inches long, for example about 25 or 50 inches long, and be less than or equal to 6 Fr. in diameter.

As embodied herein, the stabilizer member 50 can include an atraumatic tip 56 at a distal end of the stabilizer member 50. The atraumatic tip 56 can be formed as a single layer of material extending from the distal end of the stabilizer member 50. For example, the atraumatic tip 56 can be an extension of the outer layer 55 of the stabilizer member 50 beyond the other layers of the stabilizer member 50. For example, the outer layer 55 can extend about 3 mm beyond the other layers. As embodied herein, the atraumatic tip 56 can be a separate element coupled to the stabilizer member, for example, by heat bonding. The atraumatic tip 56 can reduce or prevent damage to vessel walls during delivery of the catheter 100. The atraumatic tip can also provide a distal covering for the reinforcement layer 53. The outer tubular member 20 can extend distally from the atraumatic tip 56.

As embodied herein, the stabilizer member 50 can be coupled to a strain relief 57 at a proximal end of the stabilizer member 50. The strain relief can be made of plastic, for example, polyethylene. The stabilizer member 50 and strain relief 57 can be coupled by stoppers 58a, 58b. The stoppers 58a, 58b can be shaped to allow the stabilizer member 50 to rotate about a central longitudinal axis relative the strain relief 57. For example, the stoppers 58a, 58b can be cylindrical in shape. Such a coupling can allow the stabilizer member 50 to rotate, for example, during insertion of the catheter assembly 100 and during delivery of the implant, while the handle 1 remains rotationally stationary. Alternatively, the stoppers 58a, 58b, can be shaped to prevent the stabilizer member 50 from rotating relative the strain relief 57. The strain relief 57 can be coupled to the handle 1, for example by a “maze” coupling or integrated key configurations. For example, and as embodied herein, the integrated key configuration can have a maze pattern to receive a protrusion on the housing. A detent can be provided at the end of the maze to engage the protrusion to indicate that the strain relief 57 and the handle 1 are locked together.

During operation, the strain relief 57 can keep the stabilizer member 50 axially stationary relative the handle 1. Accordingly, while the actuation assembly 2 moves the inner shaft member 30, as well as the outer tubular member 20, if so configured, relative to the handle, the stabilizer member 50 remains stationary relative to the handle. This can allow the handle to remain stationary relative the patient while the inner shaft member 30 and the outer tubular member 20 are actuated and the implant is delivered. This configuration can simplify delivery, because a physician solely needs to actuate the trigger while delivering the implant. Furthermore, the stationary stabilizer member 50 can reduce the amount of friction along the inside wall of the vessel between the entry point and the implant delivery location during operation. Such a reduction in friction can reduce the amount of force required to operate the actuation assembly 2 and move the outer tubular member 20. Additionally, the reduction in friction can reduce damage to vessel walls during operation. Alternatively, if the outer tubular member is coupled directly to the handle in a fixed relation, then the strain relief can keep the outer tubular member axially stationary relative to the handle.

As previously noted, the catheter assembly herein further includes an inner shaft member. The inner shaft member 30 as embodied herein defines an inner shaft member lumen 72. The inner shaft member 30 includes a proximal inner shaft portion 31 and a distal inner shaft portion 41 which can be formed as a single piece. Alternatively, and as embodied herein, the proximal inner shaft portion 31 can be a proximal inner shaft member (also referred to as element 31) and the distal inner shaft portion 41 can be a distal inner shaft member (also referred to as element 41). That is, the inner shaft member 30 can be composed of two separate members. As noted above, the inner shaft member 30 can be between 25 and 65 inches long, for example, the inner shaft member 30 can be about 34 or 56 inches long. As embodied herein, for illustration, the proximal inner shaft member 31 can thus be between 4 and 19 inches long, for example, about 12 inches long. The distal inner shaft member 41 can be between 14 and 51 inches long, for example, about 22 or 44 inches long. Each of the proximal inner shaft member 31 and the distal inner shaft member 41 can have an inner diameter and an outer diameter. The outer diameter 42 of the distal inner shaft member 41, or at least the proximal end thereof, can be configured to be received within the inner diameter of the proximal inner shaft member. For example, the outer diameter 42 of the distal inner shaft member 41 can be less than the inner diameter 33 of the proximal inner shaft member 31. In this manner, the outer diameter 42 of the distal inner shaft member 41 likewise will be less than the outer diameter 32 of the proximal inner shaft member 31. As embodied herein, a radiopaque marker can be disposed on the inner shaft member 30.

A proximal end portion 44 of the distal inner shaft member 41 can be coupled to a distal end portion 35 of the proximal inner shaft member 34. For example, the proximal inner shaft member 31 can be heat bonded to the distal inner shaft member 41. The proximal inner shaft member 31 and the distal inner shaft member 41 thus can together define the inner shaft member lumen 72. If the proximal end portion of the distal inner shaft member 41 is received within the inner diameter of the proximal inner shaft member 31, the inner diameter at the proximal end portion 44 of the distal inner shaft member 41 can further include a chamfer, funnel or the like 49. In this matter the chamfer 49 can facilitate delivery of a guidewire 71 through the inner shaft member lumen 72 from the proximal inner shaft member 31 to the distal inner shaft member 41.

The proximal inner shaft portion 31 and the distal inner shaft portion 41 can each be a multi-layer tube. For example, each of the proximal inner shaft portion 31 and the distal inner shaft portion 41 can include an inner layer, a reinforcement layer, and an outer layer, which can be any suitable material. For example, the inner layer 36 of the proximal inner shaft portion 31 can be a nylon, for example, rilsan aesno. The reinforcement layer 37 of the proximal inner shaft portion 31 can be a braid layer, such as a braid of SST, for example, 0.001×0.007 inch, 304V SST. Alternatively, the reinforcement layer 37 of the proximal inner shaft portion 31 can include Teflon fibers extending longitudinally along the length of the proximal inner shaft portion. The outer layer 38 of the proximal inner shaft portion 31 can be plastic, for example, grilamid. The inner layer 46 of the distal inner shaft portion 41 can be a nylon, for example a rislan aesno and nylon 12 mixture. The reinforcement layer 47 of the distal inner shaft portion 41 can be a braid layer, such as a braid of SST, for example, 0.001×0.007 inch, 304V SST. Alternatively, the reinforcement layer 47 of the distal inner shaft portion 41 can include Teflon fibers extending longitudinally along the length of the proximal inner shaft portion. The outer layer 48 of the distal inner shaft portion 41 can be plastic, for example, a grilamid and nylon 12 mixture. This three-layer design can provide improved hoop strength and flexibility, and can allow the inner tubular member to have a reduced outer diameter. Additionally, increasing the “PIC count” of the braid (i.e., providing a tighter braid) can improve pushability.

Each of the multi-layer tubular members described herein (i.e., outer tubular member, stabilizer, and inner tubular member portions, as applicable) can be made using a conventional process, such as extrusion, or be formed by a coating process if a lower profile with higher strength is needed. That is, forming each multi-layer tubular member by a coating process yields surprising performance results while providing a thin wall and suitable tolerance (i.e., outer diameter), for example, for a 6 Fr. catheter assembly having a plurality of coaxially aligned tubular members. For example, using a coating process can provide improved flexibility, deliverability, and pushability. Alternatively, each of the multi-layer tubular members described herein can be formed by other known means, for example, a film cast process, a reflow process, and coextrusion. The materials and/or surface of exposed layers of the multi-tubular members can be selected to reduce friction as particular tubular members move relative one another. For example, the outer layers 38, 48 of the proximal inner member 31 and distal inner member 41 (which can both be a plastic) can move with relatively low friction relative to the inner layer 22 of the outer tubular member 20 (which can be fluorinated ethylene propylene). Likewise, the outer layer 35 of the outer tubular member 20 (which can be a plastic) can move with relatively low friction relative the inner layer 52 of the stabilizer member (which can be polytetrafluoroethylene).

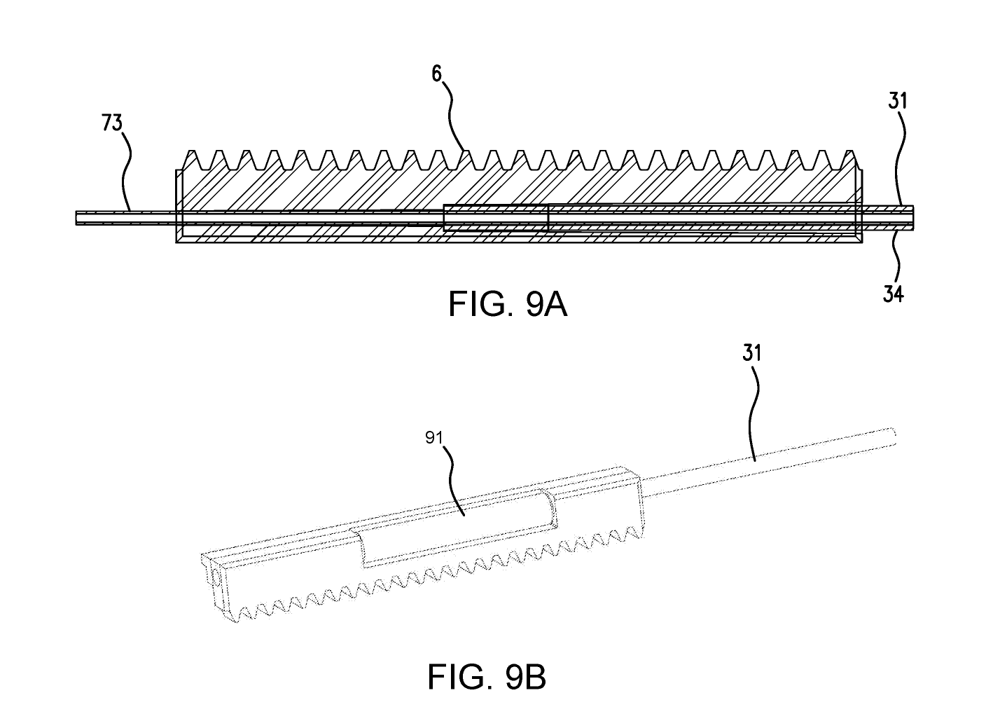

As embodied herein, a ratchet rack 6 can be coupled to the proximal end portion 34 of the proximal inner shaft portion 31. The ratchet rack 6 can be functionally coupled with the actuation assembly 2 to facilitate movement of the inner shaft member 30 proximally and distally, as described above. As with the outer tubular member, the proximal inner shaft portion 31 and ratchet rack 6 can be coupled such that the inner shaft member 30 can rotate about a longitudinal axis relative the ratchet rack 6. For example, the proximal inner shaft portion 31 and the ratchet rack can be coupled by a cylindrical coupling. Such a coupling can allow the inner shaft member 30 to rotate about a longitudinal axis, for example, during insertion of the catheter assembly 100 and during delivery of the implant, while the actuation assembly 2 remains rotationally stationary. In an alternative embodiment, the inner shaft member 30 and the ratchet rack can be coupled by a rotational sleeve 91, as shown in FIG. 9B. As a further alternative, the proximal inner shaft portion 31 can be coupled to the ratchet rack 6 such that rotation between the two members is prevented.

A hypotube 73 can be disposed at least partially within the inner shaft member lumen 72 defined by the inner shaft member 30. The hypotube 73 can extend proximally of the proximal end portion 34 of the proximal inner shaft member 31. Accordingly, the inner diameter of the proximal inner shaft member 31 can be sized to receive the hypotube 73. During operation, the hypotube 73 can act as a guiding rail for the inner shaft member 30 as the inner shaft member 30 moves proximally and distally relative to the handle 1. The inner shaft member 31 can have a sliding relationship with the hypotube 73. The hypotube 73 can be any suitable length, for example, between 15 and 25 inches, for example, 18 inches. For example, the hypotube length can be selected based on the length of the handle 1. An initial distance between a distal end of the hypotube 73, disposed within the proximal inner shaft member 31, and the proximal end portion of the distal inner shaft member 41 can be the sum of an initial length of the implant 3 and an activation length of the implant 3 (i.e., the length the implant 3 expands upon implantation). A luer 74 can be coupled to a proximal end portion of the hypotube 73.

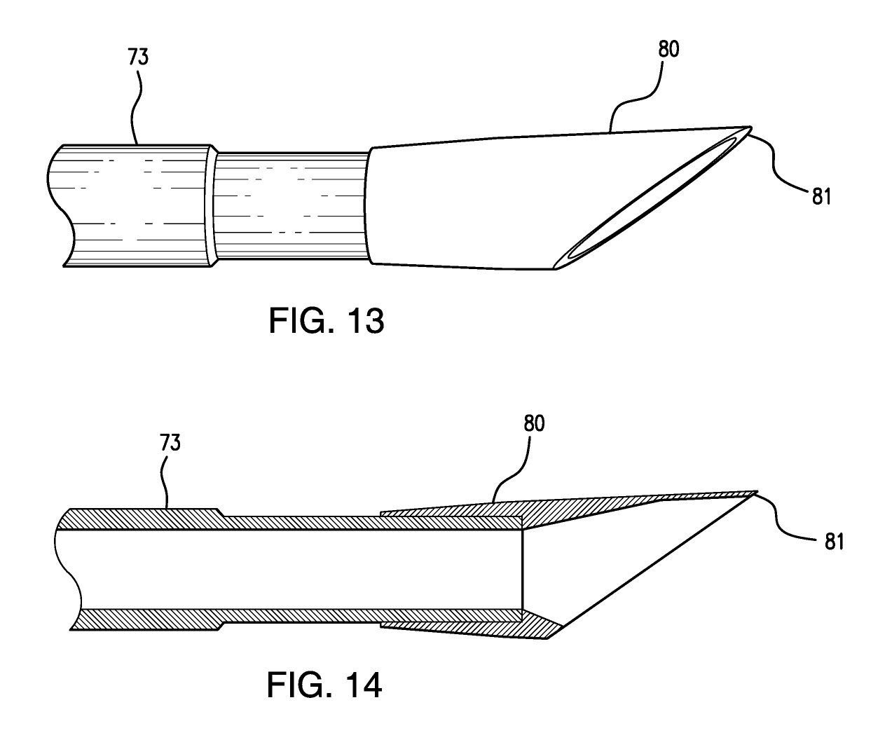

As shown in FIGS. 13-14, a sleeve 80 can be secured to a distal end of the hypotube 73 to facilitate insertion of a guidewire member through the hypotube 73 from the inner shaft member. As embodied herein, the distal end of the hypotube 73 can include a reduced diameter to receive the sleeve 680, if desired. For example, the reduced diameter can define a groove having a depth generally equal to or greater than the wall thickness of the of the sleeve. Furthermore, the sleeve 80 can have a distal end defining an angle (e.g., 30 degree angle) relative to an axis of the hypotube 73 to form a tapered distal tip 81. Additionally, the sleeve can be cylindrical with constant diameter, or generally conical in shape as shown. The sleeve 80 can be formed of any suitable material and can be selected to be relatively soft and flexible as compared to the material of the hypotube 73. As an example, and not by way of limitation, the sleeve can be a polymer material such as a composite formed of polyamide, polytetrafluoroethylene, and polyether ether ketone.

As previously noted, and as embodied herein, a pusher assembly 60 is disposed within the outer tubular member lumen 21. For purpose of illustration and not limitation, the pusher assembly 60 can have a stem 61 coupled to a distal end portion 45 of the distal inner shaft member 41, and an implant-engaging member 62 extending from the stem 61. The implant-engaging member 62 thus can be configured with a portion that extends radially outwardly and distally. The implant-engaging member 62 can be configured to engage an implant 3, for example, a stent, when distally advanced, and can be configured so as not to engage the implant 3 when proximally retracted. For example, the radially outwardly extending portion of the implant-engaging member 62 can be configured to engage one or more intersections between filaments of a woven or braided stent (e.g., a first intersection between filaments on a first side and second intersection between filaments on a second opposite side). As another example, the radially outwardly extending portion of the implant-engaging member 62 can be configured to engage one or more engageable features of other implants (for example, one or more cutouts in a laser cut stent). Additional details and examples of pusher assemblies can be found in U.S. application Ser. No. 13/118,325, filed on May 27, 2011, which is incorporated herein in its entirety. The implant 3 can be disposed within the outer tubular member lumen 21 and proximate the pusher assembly 60. The implant-engaging member 2 can have an initial positon in which the implant-engaging member 2 is disposed within the outer tubular member 21, and a deployed position in which the implant engaging member 2 extends, at least partially, distally of the outer tubular member 21. As embodied herein, a radiopaque marker can be disposed on the stem 61.

As further embodied herein, the catheter assembly can include a hollow support tube 75 disposed within the outer tubular member lumen 21 and around a least a portion of the inner shaft member 30, for example, the distal inner shaft portion 41. The support tube can be a nylon tube and can be any suitable length, for example, up to 45 inches long. Additionally or alternatively, the catheter assembly can include a support coil 76 (e.g., a support spring) or similar flexible spacing member disposed within the second lumen 21 and around at least a portion of the inner shaft member 30, for example, the distal inner shaft member 41. The support coil 76 can be any suitable length, for example about 4.5 inches long. The support tube 75 and the support coil 76 are provided and configured to reduce kinking of the catheter during delivery through a tortuous path. For example, the support tube 75 and support coil 76 can provide additional support distal of the connection between the proximal inner shaft member 31 and the distal inner shaft member 41. The support coil 76 can provide support to the outer tubular member 20 between a distal end of the support tube 75 and a proximal end of the implant 3.

Further, in accordance with the disclosed subject matter, a delivery system and method is provided incorporating the catheter assembly disclosed herein. As previously noted, the delivery system includes a handle, a trigger and an actuation assembly, as well as the catheter assembly described in detail above.

In accordance with the disclosed subject matter, the outer tubular member 20 can be fixedly coupled to a handle, and can be retracted in a proximal direction by moving the handle in a proximal direction. The trigger can advance the inner shaft member 30 distally. For example, U.S. application Ser. No. 11/876,764, filed on Oct. 22, 2007, which is incorporated by reference in its entirety herein, discloses suitable handles for the delivery system.

Alternatively, and in accordance with the disclosed subject matter, the actuation assembly 2 of the disclosed subject matter can be configured to displace the outer tubular member 20 in the proximal direction a distance first (d) relative to the handle 1 and to separately move the inner shaft member 30 distally a second distance (x) relative to the handle 1 upon deployment of the trigger 60 from the first position to the second position. Furthermore, the actuation assembly 2 is configured to move the inner shaft member 30 proximally a third distance (y) relative to the handle 1 with no displacement of the outer tubular member 20 relative to the handle 1 upon return of the trigger 60 from the second position to the first position. That is, the actuation assembly 2 can be configured to move the outer tubular member 20 in a proximal direction relative to the handle 1 and to separately move the inner shaft member 30 distally relative to the outer tubular member 20 upon deployment of the trigger 60 from the first position to the second position. The actuation assembly 2 can further be configured to move the inner shaft member 20 proximally relative to the outer tubular member 30 with no displacement of the outer tubular member 20 relative to the handle 1 upon return of the trigger 60 from the second position to the first position. Repeatedly deploying the trigger 60 from the first position to the second position and returning the trigger from the second position to the first position can cause the inner shaft member 30 to urge the implant 3 from the outer tubular member 20.

The distance (y) minus the distance (x) can be substantially equal to the distance (d). Upon deployment of the trigger 60 from the first position to the second position and return of the trigger 60 from the second position to the first position a net displacement of the inner shaft member 30 relative to the outer tubular member 20 thus can be zero. The implant 3 can have a length, and the length of the implant 3 can be greater than the distance (x). Example lengths of the implant 3, for purpose of illustration and not limitation, can be 20 mm, 30 mm, 40 mm, 60 mm, 80 mm, 100 mm, 120 mm, and 150 mm.

The distances (d), (x) and (y) can be selected based at least in part on the diameter of the implant to be delivered, the desired compression of the implant to be delivered, the path between the insertion point and the location of implant delivery, and/or other variables. As an example, and not by way of limitation, for a stent having a diameter of 4.5 mm when delivered to the vasculature, (d) can be about 12 mm, (x) can be about 28 mm, and (y) can be about 40 mm. As another example and not by way of limitation, the ratio (referred to herein as the “gear ratio”) between the net distal motion of the inner shaft member 21 relative to the outer shaft member 22 (i.e., the distance (d) plus the distance (x)) to the distance (d) can be greater than 3. As an example, the gear ratio of (12+28):(12) is about 3.3. The actuation assembly disclosed herein having such a gear ratio can be used to properly deploy a braided stent from an extended delivery configuration to an expanded deployed configuration and address a 3:1 change in length of the stent from the delivery length to the deployment length. Exemplary diameters for stents when delivered to the vasculature can range from 4 mm to 12 mm or greater. Exemplary diameters can be 4.5 mm, 5.0 mm, 5.5 mm, 6.0 mm, 6.5 mm, 7.5 mm, or 8 mm, or suitable increments therebetween. U.S. application Ser. No. 14/932,848, filed Nov. 4, 2015, U.S. application Ser. No. 14/932,795, filed Nov. 4, 2015, U.S. application Ser. No. 14/932,875, filed Nov. 4, 2015, U.S. application Ser. No. 14/932,862, filed Nov. 4, 2015, U.S. application Ser. No. 14/932,884, filed Nov. 4, 2015, U.S. application Ser. No. 14/932,805, filed Nov. 4, 2015, U.S. application Ser. No. 14/932,830, filed Nov. 4, 2015, and U.S. application Ser. No. 14/932,900, filed Nov. 4, 2015, each provide additional information regarding delivering an implant using an actuation assembly and a trigger assembly, and are each incorporated herein by reference in their entirety. The catheter system as disclosed herein can also be used with delivery systems including a user-actuatable element that allows a user to move the inner shaft member distally or proximally. Additional information on delivery systems with user-actuatable elements is provided in U.S. application Ser. No. 11/876,764, filed on Oct. 22, 2007, which is incorporated herein by reference in its entirety.

Exemplary materials of certain elements of the embodiments described herein are provided above. However, elements of the embodiments described above can be formed of any suitable materials, for example, plastics, composites, or metals. As an example and not by way of example, the strain relief can be polyethylene. The cap seal can be polycarbonate resin. The luer and ratchet rack and be can be polycarbonate resin. The hypotube and support spring can be SST.

While the disclosed subject matter is described herein in terms of certain preferred embodiments for purpose of illustration and not limitation, those skilled in the art will recognize that various modifications and improvements can be made to the disclosed subject matter without departing from the scope thereof. Moreover, although individual features of one embodiment of the disclosed subject matter can be discussed herein or shown in the drawings of one embodiment and not in other embodiments, it should be readily apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

Furthermore, it is recognized that the catheter assembly and delivery system as disclosed herein can be used in a method of delivering an implant. That is, for purpose of illustration, such method would include providing a catheter assembly as disclosed herein, positioning the distal end portion of the outer tubular member proximate a desired site, and deploying the delivery system to push the implant from the outer tubular member to the desired site.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A catheter assembly for delivering a medical device, comprising:
an outer tubular member defining an outer tubular member lumen, the outer tubular member comprising an inner layer, a reinforcement layer, a middle layer, and an outer layer;
an inner shaft member disposed at least partially within the outer tubular member lumen, the inner shaft member comprising a proximal inner shaft portion and a distal inner shaft portion, the distal inner shaft portion having a distal end portion; and
a pusher assembly coupled to the distal end portion of the distal inner shaft portion;
wherein the inner shaft member is configured to move distally and proximally relative the outer tubular member between an initial position and a deployed position.

2. The catheter assembly of claim 1, wherein the inner layer of the outer tubular member comprises fluorinated ethylene propylene.

3. The catheter assembly of claim 1, wherein the reinforcement layer of the outer tubular member comprises a stainless steel braid.

4. The catheter assembly of claim 1, wherein the reinforcement layer of the outer tubular member comprises Teflon fibers.

5. The catheter assembly of claim 1, wherein the middle layer of the outer tubular member comprise polyimide.

6. The catheter assembly of claim 1, wherein the outer layer of the outer tubular member comprises Grilamid.

7. The catheter assembly of claim 1, wherein the outer tubular member further comprises an atraumatic distal tip having a distally tapered end.

8. The catheter assembly of claim 7, wherein the atraumatic distal tip is heat bonded to the outer tubular member.

9. The catheter assembly of claim 8, wherein the atraumatic distal tip is mounted to an outer diameter of the outer tubular member.

10. The catheter assembly of claim 7, wherein the atraumatic distal tip comprises polyether block amide.

11. The catheter assembly of claim 1, further comprising a stabilizer member having a stabilizer lumen defined therethrough, the stabilizer lumen having an inner diameter sized to receive the outer tubular member therein.

12. The catheter assembly of claim 11, wherein the outer tubular member is configured to rotate about a central longitudinal axis relative to the stabilizer member.

13. The catheter assembly of claim 11, wherein the stabilizer member comprises an inner layer, a reinforcement layer, a middle layer, and an outer layer.

14. The catheter assembly of claim 11, where in the stabilizer member comprises a distal end having an atraumatic tip.

15. The catheter assembly of claim 11, further comprising a strain relief coupled to a proximal end portion of the stabilizer member.

16. The catheter assembly of claim 15, wherein the stabilizer member is configured to rotate about a central longitudinal axis relative the strain relief.

17. The catheter assembly of claim 11, wherein the catheter assembly has an outer profile less than or equal to 6 French.

18. The catheter assembly of claim 1, wherein at least one of the proximal inner shaft portion and the distal inner shaft portion comprise an inner layer, a reinforcement layer, and an outer layer.

19. The catheter assembly of claim 1, wherein the distal inner shaft portion comprises a distal inner shaft member and the proximal inner shaft portion comprises a proximal inner shaft member coupled to the distal inner shaft member.

20. The catheter assembly of claim 19, wherein a proximal end portion of the distal inner shaft member is heat bonded to a distal end portion of the proximal inner shaft member.

21. The catheter assembly of claim 19, wherein a proximal end portion of the distal inner shaft portion comprises an inner taper.

22. The catheter assembly of claim 19, wherein an outer diameter at a proximal end portion of the distal inner shaft member is sized to be received within an inner diameter at a distal end portion of the proximal inner shaft member.

23. The catheter assembly of claim 1, wherein an implant is disposed within the outer tubular member lumen proximate the pusher assembly.

24. The catheter assembly of claim 1, further comprising a support tube disposed within the outer tubular member lumen.

25. The catheter assembly of claim 24, further comprising a support coil disposed within the outer tubular member lumen distal of the support tube.

26. The catheter assembly of claim 1, wherein the pusher assembly comprises a stem coupled to the inner shaft member and an implant-engaging member extending from the stem.

27. The catheter assembly of claim 26, further comprising a guide wire lumen coupled to a distal end portion of the stem and extending distally of the outer tubular member.

28. The catheter assembly of claim 27, wherein the guidewire lumen comprises at least one radiopaque marker.

29. The catheter assembly of claim 1, further comprising a hypotube disposed at least partially within an inner shaft member lumen defined by the inner shaft member.

30. The catheter assembly of claim 29, further comprising a polymer sleeve secured to a distal end portion of the hypotube.

31. The catheter assembly of claim 29, further comprising a luer coupled to a proximal end portion of the hypotube.

32. The catheter assembly of claim 1, further comprising a ratchet rack coupled to a proximal end portion of the proximal inner shaft portion.

33. The catheter assembly of claim 1, wherein the inner shaft member is configured to rotate about a central axis relative to the actuation assembly.

34. A deliver system for delivering an implant, comprising:
a handle;
a trigger operatively coupled to the handle;
an actuation assembly operatively coupled to the trigger, and
a catheter assembly operatively coupled to the actuation assembly, the catheter assembly comprising:
an outer tubular member defining an outer tubular member lumen, the outer tubular member comprising an inner layer, a reinforcement layer, a middle layer, and an outer layer;
an inner shaft member disposed at least partially within the outer tubular member lumen, the inner shaft member comprising a proximal inner shaft portion and a distal inner shaft portion, the distal inner shaft portion having a distal end portion; and
a pusher assembly coupled to the distal end portion of the distal inner shaft portion;
wherein the actuation assembly is configured to displace the outer tubular member in the proximal direction a first distance (d) relative to the handle and to separately move the inner shaft member distally a second distance (x) relative to the handle upon deployment of the trigger from a first position to a second position, and further wherein the actuation assembly is configured to move the inner shaft member proximally a third distance (y) relative to the handle with no displacement of the outer tubular member relative to the handle upon return of the trigger from the second position to the first position.

35. The delivery system of claim 34, further comprising a stabilizer member, and wherein the catheter assembly has an outer profile less than or equal to 6 French.

36. A method of forming a catheter assembly for delivering a medical device, comprising:
forming an outer tubular member having an inner layer, a reinforcement layer, a middle layer, and an outer layer;
providing, and inserting into the outer tubular member, an inner shaft member comprising a proximal inner shaft portion and a distal inner shaft portion, each of the proximal inner shaft portion and distal inner shaft portion comprising an inner layer, a reinforcement layer, and an outer layer; and
providing, and positioning about the outer tubular member, a stabilizer member having an inner layer, a reinforcement layer, a middle layer, and an outer layer.

37. The method of claim 36, wherein forming the outer tubular member comprises forming the outer tubular member by a coating process.

38. The method of claim 36, wherein providing the stabilizer member further includes forming the stabilizer member by a coating process.

39. The method of claim 36, wherein providing the inner shaft member further includes forming the stabilizer member by a coating process.
